(19) United States
(12) Patent Application Publication
(10) Pub. No.: US 2021/0145340 A1
(43) Pub. Date: May 20, 2021

(54) INFORMATION PROCESSING SYSTEM, INFORMATION PROCESSING METHOD, AND RECORDING MEDIUM

(71) Applicant: SONY CORPORATION, TOKYO (JP)

(72) Inventor: MASAMICHI ASUKAI, TOKYO (JP)

(21) Appl. No.: 17/048,697

(22) PCT Filed: Feb. 4, 2019
(86) PCT No.: PCT/JP2019/003924

§ 371 Date: Oct. 19, 2020

(30) Foreign Application Priority Data
Apr. 25, 2018 (JP) 2018-083548

(51) Int. Cl. A61B 5/16; A61B 5/00; A61N 1/04

(52) U.S. Cl. CPC A61B 5/165; A61N 1/0408; A61B 5/7275; A61B 5/167

(57) ABSTRACT

[Problem] To provide an information processing system, an information processing method, and a recording medium capable of making a state of a user better according to an emotion of the user and improving quality of life. [Solution] The information processing system includes a control unit that estimates whether a user is positive or negative and has any one of a function of promoting an action of the user when it is estimated that the user is positive, a function of suppressing an action of the user when it is estimated that the user is negative, or a function of presenting a positive interpretation on a situation or action of the user when it is estimated that the user is negative.

### FIELD

The present disclosure relates to an information processing system, an information processing method, and a recording medium.

### BACKGROUND

Conventionally, measurement of an emotion of a user in a specific situation and estimation of a target of the emotion have been performed to provide feedback for the emotion.

For example, Patent Literature 1 below discloses that an estimated emotion of a user and an object causing the user to have the emotion are acquired, and when the estimated emotion of the user is positive, presentation information for maintaining the emotion is presented to the user, but when the estimated emotion of the user is negative, presentation information for removing the object is presented to the user.

Further, Patent Literature 2 below relates to a technique for detecting a positive emotion and a negative emotion from a content of a conversation during the conversation. Further, Patent Literature 3 below discloses a system that determines whether a mental state is positive or negative based on a facial expression. Further, Patent Literature 4 below discloses an action control system that properly learns appropriateness of an action of a robot based on an emotion change of a user by changing suitability associated with a selected action of the robot based on an emotion change of the user.

Further, Patent Literature 5 below discloses that, in a dialogue system, a negative emotion and a positive emotion are estimated from a user's utterance or motion, and a response corresponding to the emotion is made to cause the user to feel like the system sympathizes with the user, thereby building a trust between the user and the system.

### CITATION LIST

### Patent Literature

Patent Literature 1: JP 2017-201499 A

Patent Literature 2: JP 2017-091570 A

Patent Literature 3: JP 2016-147006 A

Patent Literature 4: JP 2016-012340 A

Patent Literature 5: JP 2006-178063 A

### SUMMARY

### Technical Problem

However, as for the response to an emotion of the user, only a response that is in sympathy with the emotion of the user (that is, showing empathy) or a response causing the user to maintain a positive emotion is made, and a response for making a state of the user better has not been considered.

Further, Patent Literature 1 described above discloses that, when the user has a negative emotion, information for removing an object causing the user to have the emotion is presented, but in this case, acquisition of the object is essential, and in a case where the object cannot be removed, it is difficult to reduce the negative emotion of the user.

Therefore, the present disclosure proposes an information processing system, an information processing method, and a recording medium capable of making a state of a user better according to an emotion of the user and improving quality of life.

### Solution to Problem

According to the present disclosure, an information processing system is provided that includes: a control unit that estimates whether a user is positive or negative, and has any one of a function of promoting an action of the user when it is estimated that the user is positive, a function of suppressing an action of the user when it is estimated that the user is negative, or a function of presenting a positive interpretation on a situation or action of the user when it is estimated that the user is negative.

According to the present disclosure, an information processing method, by a processor, is provided that includes: estimating whether a user is positive or negative; and performing any one of a function of promoting an action of the user when it is estimated that the user is positive, a function of suppressing an action of the user when it is estimated that the user is negative, or a function of presenting a positive interpretation on a situation or action of the user when it is estimated that the user is negative.

According to the present disclosure, a recording medium recording a program is provided that causes a computer to function as a control unit that estimates whether a user is positive or negative, and has any one of a function of promoting an action of the user when it is estimated that the user is positive, a function of suppressing an action of the user when it is estimated that the user is negative, or a function of presenting a positive interpretation on a situation or action of the user when it is estimated that the user is negative.

### Advantageous Effects of Invention

As described above, according to the present disclosure, it is possible to make a state of a user better according to an emotion of the user and improve quality of life.

Note that the above-described effect is not necessarily restrictive, and any one of effects described in the present specification or other effects that can be understood from the present specification may be exhibited in addition to or in place of the above-described effect.

### BRIEF DESCRIPTION OF DRAWINGS

FIG. 1 is a diagram for describing an overview of an information processing system according to an embodiment of the present disclosure.

FIG. 2 is a block diagram illustrating an example of a configuration of an information processing device according to a first exemplary embodiment.

FIG. 3 is a diagram illustrating an example of a data structure of an identification DB for (emotion) P/N identification according to the first exemplary embodiment.

FIG. 4 is a diagram illustrating an example of a data structure of a feedback DB for feedback selection according to the first exemplary embodiment.

FIG. 5 is a flowchart illustrating an example of an overall flow of FB processing of the information processing system according to the first exemplary embodiment.

FIG. 6 is a flowchart illustrating an example of P/N identification processing and P/N feedback selection processing according to the first exemplary embodiment.

FIG. 7 is a block diagram illustrating an example of a configuration of an information processing device according to a second exemplary embodiment.

FIG. 8 is a diagram illustrating an example of a data structure of a learning DB according to the second exemplary embodiment.

FIG. 9 is a flowchart illustrating an example of a flow of learning processing according to the second exemplary embodiment.

FIG. 10 is a block diagram illustrating an example of a configuration of an information processing device according to a third exemplary embodiment.

FIG. 11 is a flowchart illustrating an example of a flow of brain stimulation processing according to the third exemplary embodiment.

FIG. 12 is a diagram illustrating an example of a data structure of a P/N identification database according to a fourth exemplary embodiment.

FIG. 13 is a flowchart illustrating an example of a flow of feedback processing considering ethical P/N identification and legal P/N identification according to the fourth exemplary embodiment.

FIG. 14 is a diagram illustrating an example of a data structure of a feedback DB for selection of feedback including reframing FB according to a fifth exemplary embodiment.

FIG. 15 is a flowchart illustrating an example of a flow of feedback processing including reframing according to the fifth exemplary embodiment.

FIG. 16 is a diagram illustrating an example of a data structure of a database for P/N identification in consideration of ethical P/N identification and legal P/N identification according to a sixth exemplary embodiment.

FIG. 17 is a diagram illustrating an example of a data structure of a feedback DB for selection of feedback including reframing FB according to the sixth exemplary embodiment.

### DESCRIPTION OF EMBODIMENTS

Hereinafter, a preferred embodiment of the present disclosure will be described in detail with reference to the appended drawings. Note that, in the present specification and the drawings, components having substantially the same functional configuration are provided with the same reference signs, so that repeated description of these components is omitted.

Further, the description will be made in the following order.

1. Overview of Information Processing System According to Embodiment of Present Disclosure

2. Exemplary Embodiments

2-1. First Exemplary Embodiment (Promotion and Suppression of Action)

(2-1-1. Configuration Example)

(2-1-2. Operation Processing)

2-2. Second Exemplary Embodiment (Learning in Database)

(2-2-1. Configuration Example)

(2-2-2. Operation Processing)

2-3. Third Exemplary Embodiment (Brain Stimulation)

(2-3-1. Configuration Example)

(2-3-2. Operation Processing)

2-4. Fourth Exemplary Embodiment (Ethical and Legal Considerations)

(2-4-1. Configuration Example)

(2-4-2. Operation Processing)

2-5. Fifth Exemplary Embodiment (Reframing)

(2-5-1. Configuration Example)

(2-5-2. Operation Processing)

(2-5-3. Adding Response Showing Empathy)

(2-5-4. Automatic Generation of Reframing)

2-6. Sixth Exemplary Embodiment (Example of Application to Small Communities)

(2-6-1. Configuration Example)

(2-6-2. Operation Processing)

3. Conclusion

### 1. Overview of Information Processing System According to Embodiment of Present Disclosure

An information processing system according to the present embodiment is an agent system that estimates an emotion of a user based on an action of the user or a situation, and gives a feedback corresponding to the estimated emotion, thereby further improving a life of the user, that is, improving quality of life (QOL) of the user. It is necessary to have a negative emotion such as anger or sadness toward an environment, situation, or people, in order to detect a danger and avoid the danger, but an excessive negative emotion leads to stress and may adversely affect an immune system or the like. On the other hand, having a positive emotion has a good influence on the immune system or the like, and can be said as a more favorable state.

(Background)

In recent years, a voice agent that recognizes an uttered voice of the user and directly responds to a user's question or request in one short-term session (completed with a request and a response) has become popular. Such a voice agent is installed, for example, as a home agent for domestic use, in a dedicated speaker device (so-called home agent device) placed in a kitchen, a dining room, or the like.

Further, in the above-described existing techniques, a voice agent that makes a response corresponding to an estimated emotion of the user is also proposed.

However, in all of the techniques, the system expresses the same emotion as that of the user, thereby showing empathy to the user, and improvement (including long-term state improvement such as improvement of a living, a life, or the like) of a state of the user regardless of positive/negative has not been sufficiently considered.

In this regard, according to the present embodiment, it is possible to make a state of a user better and improve quality of life by providing feedback corresponding to an emotion of the user.

Specifically, the information processing system (agent system) according to the present embodiment recognizes an action of the user in a specific situation, estimates an emotion at that time, and gives feedback for promoting (increasing the action) or suppressing (reducing the action) the action at that time according to the estimated emotion, thereby further improving a life of the user. For example, in a case where the emotion of the user is positive, the information processing system according to the present embodiment gives positive feedback (a positive reinforcer based on behavior analysis) for promoting the action at that time to increase the action. Further, in a case where the emotion of the user is negative, the information processing system according to the present embodiment gives negative feedback (a negative reinforcer based on behavior analysis) for suppressing the action at that time to reduce the action. Further, the information processing system according to the present embodiment can also turn the emotion of the user into positive by performing reframing that presents a positive interpretation with respect to a situation or action that the user perceives as negative.

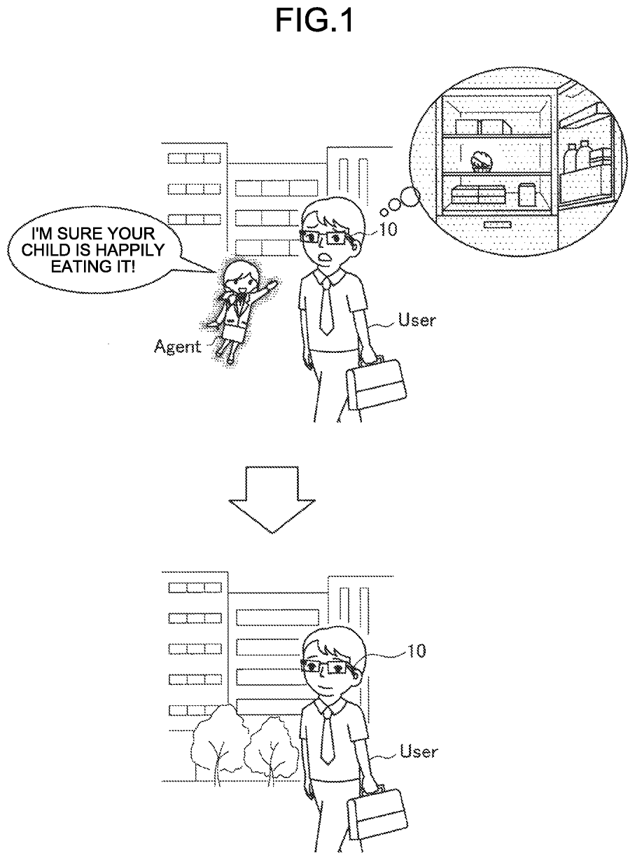

Here, FIG. 1 illustrates a diagram for describing an overview of the information processing system according to the present embodiment. FIG. 1 is a diagram for describing an example of the reframing. As illustrated in FIG. 1, for example, when the user forgot to eat a dessert (for example, a food with a short shelf life such as cream puff) before going out, and thus has a negative emotion, the agent presents a positive interpretation such as “I'm sure your child is happily eating it!” or “I'm sure your child will find it and eat it, thinking dad left it for me!”, thereby making it possible to turn a state of the user into a positive state without changing the situation or action. For the reframing performed by the agent, for example, only a sound may be presented from an earphone of a head mounted display (HMD) (information processing device 10) worn by the user, or a video of an agent character or the like may be displayed in augmented reality (AR) on a display unit of the HMD.

The recognition of the situation or action of the user, and the emotion estimation can be performed by using various sensing data, information (schedule information, a mail, a posted message on a social network, or the like) input by the user, information (date, map, weather, surveillance footage, or the like) acquired from the outside, or machine learning. A specific example of the sensing data will be described later. For example, information sensed by a sensor such as a camera, a microphone, an acceleration sensor, a gyro sensor, a location information acquisition unit, a biological sensor (for example, body temperature, pulse, heartbeat, sweating, blink, or brain wave), a gaze sensor, or an environmental sensor (for example, temperature, humidity, illuminance, or wind pressure) can be assumed. These sensors may be provided in, for example, the information processing device 10, or may be implemented by another wearable device, a smartphone, or the like that is communicatively connected to the information processing device 10.

Further, in a case where the user forgot to eat a dessert before going out and has a negative emotion as assumed in FIG. 1, for example, first, a fact that the user purchased the dessert and put the dessert in a refrigerator, an expiration date of the dessert, and the like can be acquired from a shopping record (for example, credit card statement or electronic money statement) of the user, a camera of the refrigerator, a camera of a smartphone, a posting on a social network, and the like. Then, a fact that the user is on the way to work can be acquired from a location or movement of the user, a date and time, and the like. As a result, it is possible to understand that the user left the dessert at home (situation recognition) and forgot to eat before going out (action recognition). In a case where the user has a negative emotion due to the action in such a situation, as described above, it is possible to turn the state of the user to a positive state by presenting a positive interpretation such as “I'm sure your child is happily eating it!”.

Whether or not the user has a negative emotion may be identified based on, for example, a database in which emotions (negative/positive) are associated with situations and actions, the database being prepared in advance. Further, a timing for the reframing may be when the user takes an action such as “going out”, when a stomach growls (rumbling), when the user mumbles something about forgetting to eat the dessert, when the user posts on a social network, when the user sighs while keeping eyes on a food shop, a food sign, or the like, when the user walks with his/her head down (it can be estimated that the user is depressed), when it is estimated from an eye movement, brain waves, or the like that the user is thinking of something, when it is estimated from a blood sugar level, brain waves, and the like that the user is hungry or is thinking about food, or the like.

The information processing system according to the embodiment of the present disclosure has been described hereinabove. The information processing system (also referred to as agent system) according to the present embodiment is implemented by various devices. For example, the agent system according to the present embodiment can be implemented by an output device such as a smartphone, a tablet terminal, a mobile phone, a dedicated terminal such as a home agent (speaker device), a projector, or a wearable device such as a head mounted display (HMD), a smart band, a smart watch, or a smart ear mounted on the ear. The HMD may be, for example, a spectacle-type HMD that includes earphones and a see-through display unit and can be worn on a daily basis (see FIG. 1).

Further, the agent system according to the present embodiment is an application executed on these output devices or a server, and may be implemented by a plurality of devices. Further, in the agent system according to the present embodiment, an arbitrary output device may provide feedback as appropriate. For example, while the user is at home, a speaker device or display device (a TV receiver, a projector, a large display device installed in a room, or the like) in the room mainly provides the feedback, but while the user is outside the home, a smartphone, a smart band, a smart earphone, or the like mainly provides the feedback. Further, the feedback may be provided by a plurality of devices at substantially the same time.

Further, the feedback (promotion, suppression, and reframing) to the user can be provided in a form of sound output, image display, text display, tactile stimulation on the body, brain stimulation, or the like.

Subsequently, the information processing system according to the present embodiment will be described in detail by using a plurality of exemplary embodiments.

### 2. Exemplary Embodiments

### 2-1. First Exemplary Embodiment (Promotion and Suppression of Action)

### 2-1-1. Configuration Example

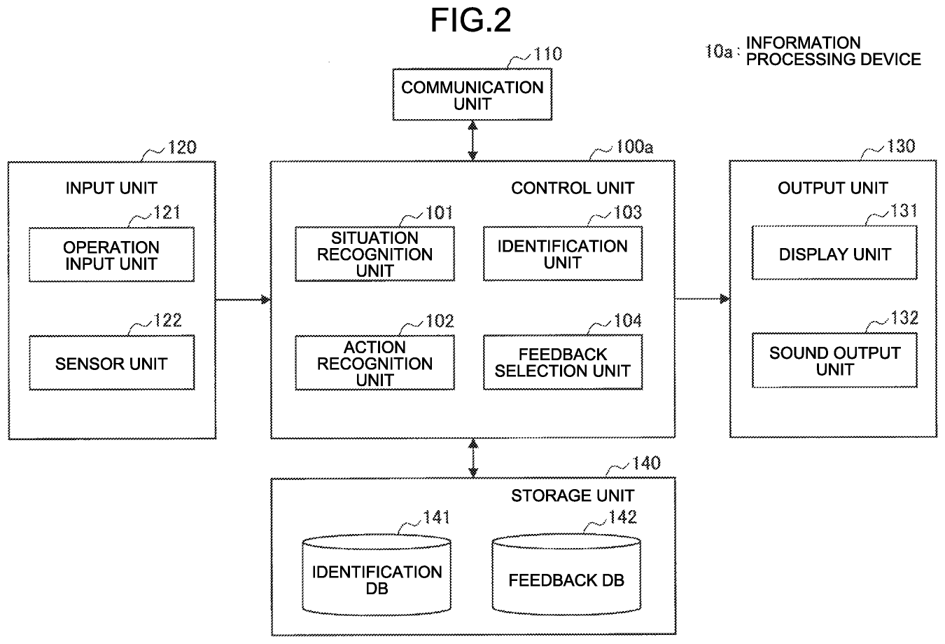

FIG. 2 is a block diagram illustrating an example of a configuration of an information processing device 10a according to a first exemplary embodiment. As illustrated in FIG. 2, the information processing device 10a includes a control unit 100a, a communication unit 110, an input unit 120, an output unit 130, and a storage unit 140.

The control unit 100a functions as an arithmetic processing device and a control device, and controls an overall operation in 10a according to various programs. The control unit 100a is implemented by an electronic circuit such as a central processing unit (CPU) or a microprocessor, for example. Further, the control unit 100a may include a read only memory (ROM) that stores programs, arithmetic operation parameters, or the like to use, and a random access memory (RAM) that temporarily stores parameters varying as appropriate, or the like.

Further, the control unit 100a according to the present embodiment also functions as a situation recognition unit 101, an action recognition unit 102, an identification unit 103, and a feedback selection unit 104.

The situation recognition unit 101 recognizes an environment in which the user is placed as a situation. Specifically, the situation recognition unit 101 recognizes a user situation based on sensing data (sound, camera video, biological information, motion information, or the like) sensed by a sensor unit 122. For example, the situation recognition unit 101 recognizes a vehicle (train, bus, car, bicycle, or the like) used by the user based on a location of the user, a moving speed, and acceleration sensor data. Further, for the situation recognition, the sensor unit 122 may label an environment with an AND condition of language (for example, “train & crowded” (crowded train) or “room & child” (child's room)).

Further, the situation recognition unit 101 may also recognize a situation that the user actually perceives (experiences). For example, the situation recognition unit 101 can recognize the situation that the user actually perceives by using only a captured image (for example, a camera provided in the HMD and having an image capturing view angle corresponding to a visual field of the user) obtained by capturing an image of an area in a gaze direction of the user, or sound data collected by a microphone positioned near the ear of the user. Alternatively, in order to limit the situation to only a situation where the user pays attention, the situation recognition unit 101 may exclude data when the eyes are closed for a long time by sensing opening/closing of the eyes, or exclude data other than that when the user pays attention (concentration) by sensing a brain activity such as brain waves.

Further, the situation recognition unit 101 may recognize the situation by referring to information (schedule information, a mail content, a content posted on a social network, or the like) input by the user, information (a date and time, weather, traffic information, a user's purchase history, sensing data acquired from a sensor device (surveillance camera, surveillance microphone, or the like) installed in the vicinity, or the like) acquired by the information processing device 10a, or the like, in addition to the sensing data sensed by the sensor unit 122.

In addition, the situation recognition unit 101 may also recognize a situation (for example, interaction in a music group in which the user participates, browsing, or downloading) on a social network or a situation in a virtual reality (VR) world, in addition to a situation in the real world.

Further, the situation recognition unit 101 may use, as a situation recognition method, a neural network trained by deep learning by giving sensing data and a language label as trainer data.

The action recognition unit 102 recognizes the action of the user. Specifically, the action recognition unit 102 recognizes a user action based on sensing data (sound, camera video, biological information, motion information, or the like) sensed by the sensor unit 122. For example, the action recognition unit 102 recognizes an action such as “stand, sit, walk, run, lie, fall, or talk” in real time based on sensing data sensed by an acceleration sensor, a gyro sensor, a microphone, or the like.

The identification unit 103 identifies a user emotion based on the user situation recognized by the situation recognition unit 101 and the user action recognized by the action recognition unit 102. The “emotion” may be expressed as a basic emotion such as joy or anger, but here, as an example, the emotion may be expressed as positive/negative (hereinafter, also referred to as P/N). The positive/negative emotion corresponds to, for example, an axis of “pleasure/misery” in the Russell's circular model that organizes human emotions on two axes: “arousal” and “pleasure/misery (valence)”. Examples of the positive emotion include joy, happiness, excitement, relaxation, and satisfaction. Examples of the negative emotion include anxiety, anger, dissatisfaction, irritation, discomfort, sadness, depression, and boredom. Note that a level of the positive or negative emotion may be expressed by valence and normalized from −1 to 1. An emotion having valence of “−1” is the negative emotion, an emotion having valence of “0” is a neutral emotion, and an emotion having valence of “1” is the positive emotion.

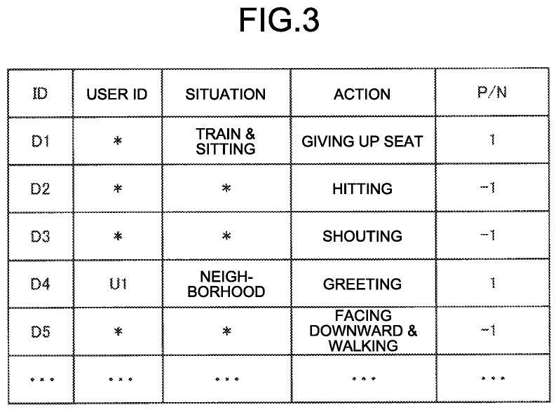

The identification unit 103 according to the present embodiment may identify the user emotion (P/N) based on the user action and the user situation by using an identification database (DB) 141 for P/N identification, generated in advance. Here, FIG. 3 illustrates an example of a data structure of the identification DB 141 for (emotion) P/N identification according to the present embodiment. As illustrated in FIG. 3, the identification DB 141 stores data in which (emotion) P/N is associated in advance with a situation and an action (1: positive and −1: negative). Therefore, the identification unit 103 identifies whether the user emotion is positive or negative based on the situation and action of the user. A mark “*” in the table illustrated in FIG. 3 means “don't care”. In a case where a user ID is “*”, data can be applied to anyone, and in a case where the situation/action is “*”, data can be applied to any situation/action. P/N identification data accumulated in the identification DB 141 may be, for example, general knowledge, a social common notion, or data generated in advance based on a questionnaire result regarding likes and dislikes or preference of the user, or may be automatically generated by learning to be described later.

Referring to the example illustrated in FIG. 3, the identification unit 103 estimates that “P/N: 1 (positive)” in a case where any user takes an action to give up his/her seat to someone in a situation where the user sits on a train (specifically, a case of a change from a sitting state to a standing state on the train, or the like). Further, referring to the example illustrated in FIG. 3, the identification unit 103 estimates that “P/N: −1 (negative)” in a case of recognizing that any user takes an action such as “shouting” or “hitting” in any situation.

Note that in the example illustrated in FIG. 3, the positive emotion and the negative emotion are represented by discrete values of “1” and “−1”, respectively, by way of example, but the present embodiment is not limited thereto, and for example, the positive emotion and the negative emotion may be represented by continuous values.

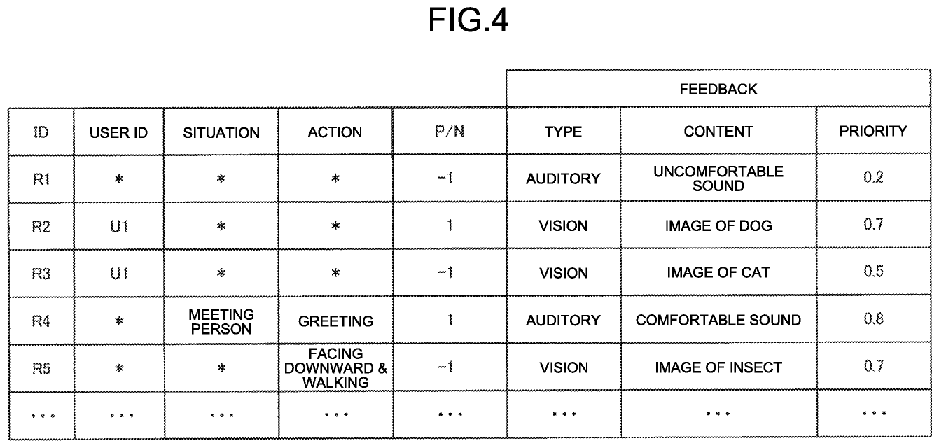

The feedback selection unit 104 has a function of selecting feedback to the user based on an identification result of the identification unit 103. Specifically, the feedback selection unit 104 selects feedback (hereinafter, also referred to as “FB”) to the user by using a feedback DB 142 based on the identification result of the identification unit 103. Here, FIG. 4 illustrates an example of a data structure of the feedback DB 142 for feedback selection according to the present embodiment. As illustrated in FIG. 4, in the feedback DB 142, information regarding an FB content (a type, content, and priority of FB) associated in advance with a user ID, a situation, an action, and (emotion) P/N is stored. The FB type may be tactile stimuli (electricity, temperature, wind, pressure, and the like), olfaction (smell), and the like in addition to auditory stimuli (sound) and vision (video) illustrated in FIG. 4. The feedback selection unit 104 selects an output method (sound output, display output, tactile stimulus output, or the like) according to the FB type. Further, in a case where there are a plurality of output devices, the feedback selection unit 104 can select an appropriate output device according to the FB type. Note that feedback data accumulated in the feedback DB 142 is, for example, general knowledge, a social common notion, or data generated in advance based on a questionnaire result regarding likes and dislikes or preference of the user.

For a positive action, FB that is pleasant to the user is provided in order to reinforce the action, and for a negative action, FB that is unpleasant to the user is provided in order to suppress the action. Only one FB or a plurality of FBs may be provided. When being selected from a plurality of FB candidates, FB may be selected according to the priority illustrated in FIG. 4. The priority illustrated in FIG. 4 is set in advance.

In the example illustrated in FIG. 4, for example, it is known from the questionnaire result acquired in advance that a user with a user ID U1 likes dogs and dislikes cats, therefore, for an action when it is identified that the emotion is positive, a bark or animation of a dog that the user likes is presented to lift the user's mood and reinforce the action. On the other hand, for an action when it is identified that the emotion is negative, a sound or animation of a cat that the user dislikes is presented. In this case, although the user is temporarily offended, as the action is reduced in future, the state of the user can become better in the long-term perspective. Further, a “pleasant sound” illustrated in FIG. 4 is assumed to be music that offers a relaxing effect, happiness, and satisfaction such as music (melody) that is generally considered to be comfortable, or a babbling sound of a stream. Alternatively, the “pleasant sound” may be a sound of an animal or character that the user likes, music that the user likes, or the like. On the other hand, an “unpleasant sound” may be a sound that generally makes people feel uncomfortable (for example, a sound made by a nail scratching on a blackboard, a high-pitched metallic sound, a warning sound, a sound of a specific frequency, or the like) or a sound that the user personally feels uncomfortable (a sound effect such as a sound made when an expanded polystyrene is rubbed, a sound of an animal or character that the user dislikes, music that the user dislikes, or the like).

(Communication Unit 110)

The communication unit 110 is connected to an external device in a wired or wirelessly manner, and transmits and receives data to and from the external device. The communication unit 110 is communicatively connected to the external device through a network, for example, a wired/wireless local area network (LAN), Wi-Fi (registered trademark), Bluetooth (registered trademark), a mobile communication network (long term evolution (LTE) or 3rd generation mobile communication system (3G)), or the like.

(Input Unit 120)

The input unit 120 acquires input information for the information processing device 10a and outputs the input information to the control unit 100a. The input unit 120 includes, for example, an operation input unit 121 and the sensor unit 122.

The operation input unit 121 detects information on an operation input by the user with respect to the information processing device 10a. The operation input unit 121 may be, for example, a touch sensor, a pressure sensor, or a proximity sensor. Alternatively, the operation input unit 121 may be a physical component such as a button, a switch, or a lever.

The sensor unit 122 is a sensor that senses various types of sensing data for recognizing a user situation or a user action. The sensor unit 122 is assumed to be, for example, a camera (stereo camera, visible light camera, infrared camera, depth camera, or the like), a microphone, a gyro sensor, an acceleration sensor, a geomagnetic sensor, a biological sensor (heartbeat, body temperature, sweating, blood pressure, pulse, respiration, gaze, blink, eye movement, gaze duration, brain waves, body movement, body position, skin temperature, skin electrical resistance, microvibration (MV), myogenic potential, SpO2 (blood oxygen saturation), or the like), a location information acquisition unit of a global navigation satellite system (GNSS) or the like, an environment sensor (illuminance sensor, atmospheric pressure sensor, temperature (air temperature) sensor, humidity sensor, or altitude sensor), an ultrasonic sensor, or an infrared sensor. In addition to the GNSS, the location information acquisition unit may sense a location by using Wi-Fi (registered trademark), Bluetooth (registered trademark), performing transmission and reception with a mobile phone, a personal handyphone system (PHS), a smartphone, or the like, or using near-field communication, for example. The number of respective sensors may be plural. Further, the microphone may be a directional microphone.

(Output Unit 130)

The output unit 130 has a function of outputting feedback to the user under the control of the control unit 100a. The output unit 130 includes, for example, a display unit 131 and a sound output unit 132. The display unit 131 is a display device that displays an image (still image or moving image) or text. The display unit 131 is implemented by, for example, a display device such as a liquid-crystal display (LCD) or an organic electroluminescence (EL) display. The output unit 130 may be, for example, a see-through display unit provided in a spectacle-type HMD. Here, display information such as a feedback image is displayed in augmented reality (AR) by being superimposed on a real space. Further, the sound output unit 132 outputs an agent voice, music, a melody, a sound effect, and the like. The number of display units 131 and the number of sound output units 132 may be plural. Further, the sound output unit 132 may be a directional speaker.

(Storage Unit 140)

The storage unit 140 is implemented by a read only memory (ROM) that stores programs, arithmetic operation parameters, or the like to be used in processing performed by the control unit 100a, and a random access memory (RAM) that temporarily stores parameters varying as appropriate, or the like.

Further, the storage unit 140 stores the identification DB 141 and the feedback DB 142. The identification DB 141 includes data for identifying whether the user emotion is negative or positive based on a situation recognition result and an action recognition result as described with reference to FIG. 3. Further, the feedback DB 142 includes data for selecting feedback to the user based on a P/N identification result as described with reference to FIG. 4. In the present embodiment, as an example, based on behavior analysis, a positive reinforcer (reward) as positive FB for action promotion, and a negative reinforcer (penalty) as negative FB for action suppression are accumulated.

The configuration of the information processing device 10a according to the present embodiment has been specifically described above. Note that the configuration of the information processing device 10a is not limited to the example illustrated in FIG. 2. The information processing device 10a may be implemented by a plurality of devices. For example, at least a part of the information processing device 10a may be provided in a server on the network. Specifically, each function of the control unit 100a of the information processing device 10a and each DB of the storage unit 140 may be provided in the server. Further, the sensor unit 122 may be provided in the external device, or may be provided in both the information processing device 10a and the external device.

Further, FIG. 2 illustrates specific examples of the input unit 120 and the output unit 130, but the present embodiment is not limited thereto. Further, the information processing device 10a may have a configuration in which the output unit 130 does not include the display unit 131, or a configuration in which the output unit 130 does not include the sound output unit 132. In addition, the information processing device 10a may have any one of an action promoting FB function or an action suppressing FB function.

### 2-1-2. Operation Processing

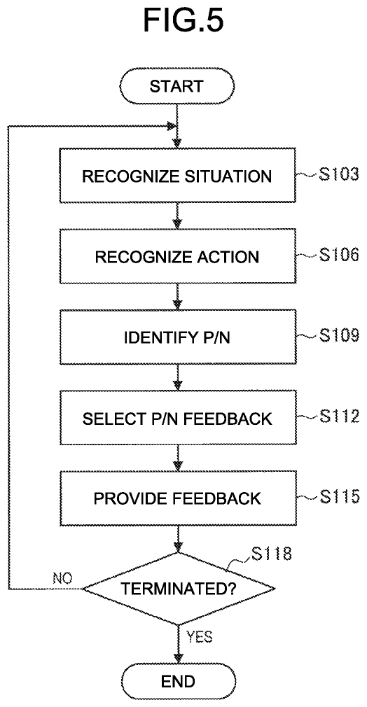

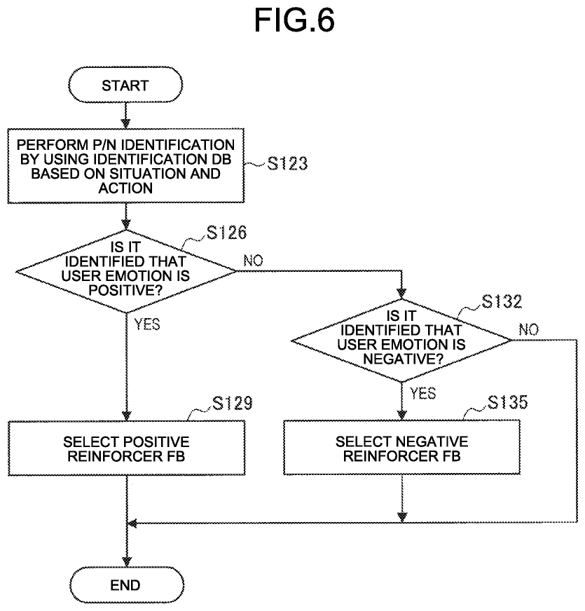

Next, FB processing of the information processing system according to the present embodiment will be specifically described with reference to FIGS. 5 and 6.

FIG. 5 is a flowchart illustrating an example of an overall flow of the FB processing of the information processing system according to the present embodiment.

As illustrated in FIG. 5, first, the information processing device 10a performs situation recognition (Step S103) and action recognition (Step S106). The situation recognition and the action recognition are not limited to the order of the flow illustrated in FIG. 5, and may be performed in the reverse order or in parallel. Further, the situation recognition and the action recognition can be performed continuously/periodically.

Next, the information processing device 10a refers to the identification DB 141 to perform P/N identification based on a situation recognition result and an action recognition result (Step S109).

Next, the information processing device 10a refers to the feedback DB 142 to select feedback based on a P/N identification result (Step S112).

Then, the information processing device 10a provides the selected feedback (Step S115).

The above-described processing in Steps S103 to S115 are repeated until the system is terminated (for example, an explicit termination instruction from the user) (Step S118).

Next, a flow of P/N identification processing and P/N feedback selection processing will be described with reference to FIG. 6. FIG. 6 is a flowchart illustrating an example of the P/N identification processing and the P/N feedback selection processing.

As illustrated in FIG. 6, first, the identification unit 103 of the information processing device 10a performs P/N identification by using the identification DB 141 based on the situation recognition result and the action recognition result (Step S123).

Next, in a case where the identification unit 103 identifies that the user emotion is positive (Step S126/Yes), the feedback selection unit 104 selects positive reinforcer FB (Step S129). For example, in a case where the user U1 takes a “greeting” action in a situation of “meeting person” and it is identified that the user emotion is positive, feedback corresponding to R2 (ID) and feedback corresponding to R4 (ID) among FBs illustrated in FIG. 4 are suitable. The feedback selection unit 104, for example, presents a video in which a dog is waving its tail happily on the display unit 131 of the see-through HMD worn by the user U1, or plays a comfortable sound from the sound output unit 132 of the HMD. In a case where only one feedback is provided, only R4 (ID) with a higher priority is fed back. As such, as the positive reinforcer FB, a pleasant sound is played, a pleasant vibration is applied, a favorite music is played, a video of a favorite animal is played, or a favorite character says a positive word, such that the user's mood is lifted, and the “greeting” action is reinforced.

On the other hand, in a case where it is identified that the user emotion is negative (Step S132/Yes), the feedback selection unit 104 select negative reinforcer FB (Step S135). For example, a case where a user U2 is walking with his/her head down will be described. The situation recognition unit 101 recognizes, from location information of the user U2, a situation of “commuting” in which the user U2 is moving between his/her home and a work place, and the action recognition unit 102 recognizes from an acceleration sensor that the user U2 is walking and recognizes from an acceleration sensor on the head that the user U2 is facing downward, thereby recognizing that the user U2 is walking with his/her head down.

The identification unit 103 identifies whether the action of walking with his/her head down during commuting is positive or negative. Specifically, the identification DB 141 is referred to. Since it is easy to feel depressed when walking with his/her head down, as indicated in D5 (ID) of FIG. 4, the action of “walking with his/her head down” corresponds to −1 (P/N), that is, the action of “walking with his/her head down” is associated with the negative emotion in advance in the identification DB 141. Therefore, the identification unit 103 identifies that the user emotion is negative based on D5 (ID). Next, since the user U2 takes the action of “facing down & walking” in the situation of “commuting” and it is identified that the user emotion is negative (P/N: −1), the feedback selection unit 104 refers to the example of the feedback DB 142 illustrated in FIG. 4 and selects P/N feedback corresponding to R1 (ID) and P/N feedback corresponding to R5 (ID). The feedback selection unit 104, for example, presents a video in which a generally disliked insect is moving on the display unit 131 of the see-through HMD worn by the user U2, or plays an uncomfortable sound from the sound output unit 132 of the HMD. In a case where only one feedback is provided, only R5 (ID) with a higher priority is fed back.

As such, as the negative reinforcer FB, an unpleasant sound is played, an unpleasant vibration is applied, a video of a disliked animal is played, or a favorite character says a negative word, such that the action of “walking with his/her head down” of the user U2 can be weakened, that is, suppressed. In addition, when walking while keeping his/her head up, reinforcement such as playing a favorite music is performed as feedback, the action of walking while keeping his/her head up is promoted, and the action of walking with his/her head down can be further suppressed.

Note that, since there is a case where the emotion is neutral, no feedback is provided in a case where the identification result does not correspond to either positive or negative (Step S132/No).

### 2-2. Second Exemplary Embodiment (Learning in Database)

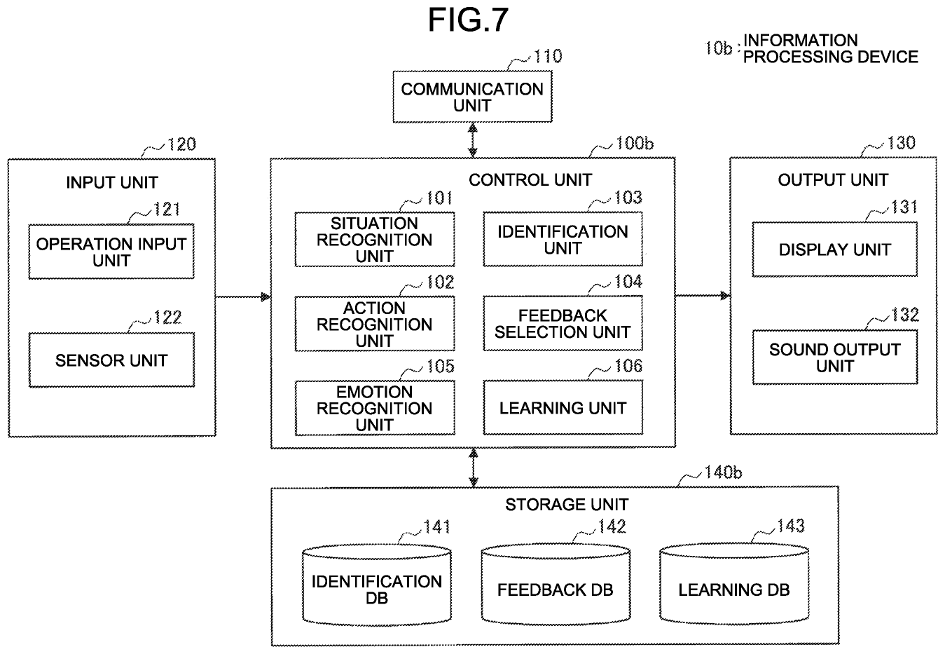

Next, a second exemplary embodiment will be described with reference to FIGS. 7 to 9. The data of the identification DB 141 and the feedback DB 142 used in the above-described first exemplary embodiment can also be generated by learning.

More specifically, in the information processing system according to the present exemplary embodiment, a user emotion is recognized based on sensing data of the user, and an emotion recognition result is recorded in a database together with a situation and an action at that time, such that data learning in the identification DB 141 and the feedback DB 142 is performed. Hereinafter, a configuration and operation processing of an information processing device 10b according to the present exemplary embodiment will be specifically described.

### 2-2-1. Configuration Example

FIG. 7 is a block diagram illustrating an example of a configuration of the information processing device 10b according to the present exemplary embodiment. As illustrated in FIG. 7, the information processing device 10b includes a control unit 100b, a communication unit 110, an input unit 120, an output unit 130, and a storage unit 140b. Note that contents of the configuration denoted by the same reference numerals as the configuration described with reference to FIG. 2 are as described above, and thus a description thereof is omitted here.

The control unit 100b functions as an arithmetic processing device and a control device, and controls an overall operation in 10b according to various programs. Further, the control unit 100b also functions as a situation recognition unit 101, an action recognition unit 102, an identification unit 103, a feedback selection unit 104, an emotion recognition unit 105, and a learning unit 106.

The storage unit 140b stores an identification DB 141, a feedback DB 142, and a learning DB 143.

The emotion recognition unit 105 recognizes a user emotion based on sensing data acquired by a sensor unit 122 and the like. For example, the emotion recognition unit 105 may recognize the emotion by analyzing a facial expression in a captured image obtained by capturing an image of a face of the user, or may recognize the emotion by analyzing biological sensor data such as heartbeat or pulse. An algorithm of the emotion recognition is not particularly limited. Further, in the emotion recognition, the emotion may be expressed as a basic emotion such as joy or anger. However, similarly to the first exemplary embodiment described above, the emotion may also be expressed by valence as an example and normalized from −1 to 1. An emotion having valence of “−1” is the negative emotion, an emotion having valence of “0” is a neutral emotion, and an emotion having valence of “1” is the positive emotion. For example, the emotion recognition unit 105 compares a value of the biological sensor data with a predetermined threshold value to calculate the valence (pleasure/misery), thereby recognizing the emotion. Note that, here, a case where emotions are represented by discrete values such as “−1”, “0”, and “1”, respectively, has been described by way of example, but the present embodiment is not limited thereto, and the emotions may be represented by continuous values. For example, sensing data (analog value) such as the biological sensor data may be accumulated and quantified as a value of the emotion.

An emotion recognition result obtained by the emotion recognition unit 105 is accumulated in the learning DB 143 together with a situation recognition result obtained by the situation recognition unit 101 and an action recognition result obtained by the action recognition unit 102 at the same time.

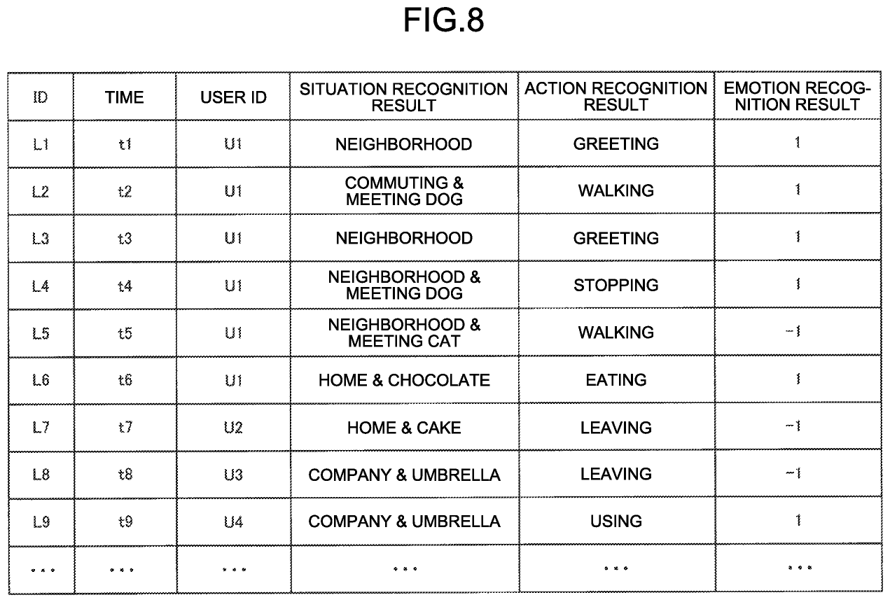

In the learning DB 143, the situation recognition result obtained by the situation recognition unit 101, the action recognition result obtained by the action recognition unit 102, and the emotion recognition result obtained by the emotion recognition unit 105 are accumulated together with time. Here, an example of a data structure of the learning DB 143 is illustrated in FIG. 8. In the example illustrated in FIG. 8, the emotion recognition result is expressed by valence. As illustrated in FIG. 8, for example, it is recorded that an emotion recognition result when a user U1 takes a “walking” action or a “stopping” action in a situation of “meeting dog during commuting” is “valence: 1” (that is, a positive emotion). On the other hand, it is recorded that an emotion recognition result when the user U1 takes the “walking” action in a situation of “meeting cat during commuting” is “valence: −1” (that is, a negative emotion). The data stored in the learning DB 143 are used when the learning unit 106 performs the data learning in the identification DB 141 or the data learning in the feedback DB 142.

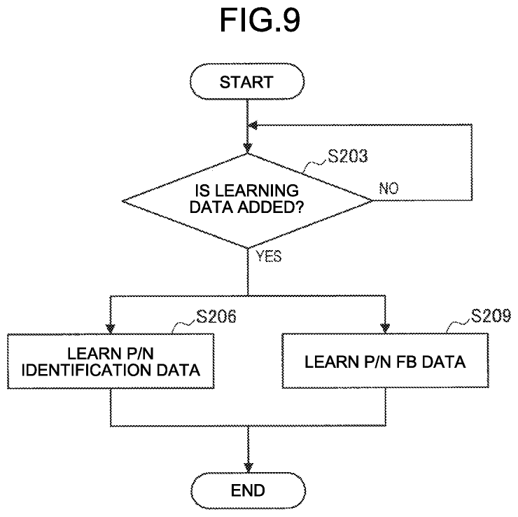

The learning unit 106 can perform the data learning in the identification DB 141 and the data learning in the feedback DB 142 by using the data accumulated in the learning DB 143. Details will be described in a description of a flowchart illustrated in FIG. 9 below.

### 2-2-2. Operation Processing

FIG. 9 is a flowchart illustrating an example of a flow of the learning processing according to the present exemplary embodiment. As illustrated in FIG. 9, the learning unit 106 determines whether or not learning data is added to the learning DB 143 (Step S203), and in a case where the learning data is added (Step S203/Yes), P/N identification data learning in which data is added to the identification DB 141 (Step S206) or P/N FB data learning in which data is added to the feedback DB 142 (Step S209) is performed. Note that the learning performed by the learning unit 106 may be performed each time the learning data is added to the learning DB 143, or may be performed based on a newly added learning data at regular intervals.

First, a specific example of the P/N identification data learning in the identification DB 141 will be described using the table illustrated in FIG. 8. For example, in a case where the number of times the emotion of the user U1 becomes positive when greeting in the neighborhood like L1 (ID) and L3 (ID) illustrated in FIG. 8 is more than a certain number of times, the learning unit 106 generates data indicating that the emotion of the user U1 becomes positive when greeting in the neighborhood, and adds the data to the identification DB 141. For example, the learning unit 106 adds, as P/N identification data, [user ID: U1, situation: neighborhood, action: greeting, P/N: 1] as indicated in D4 (ID) in FIG. 3.

Further, in a case where P/N identification data [U2, neighborhood, greeting, 1] indicating that the emotion of another user, for example, the user U2, also becomes positive when greeting in the neighborhood already exists when such P/N identification data of the user U1 is added, the learning unit 106 may generalize these two P/N identification data, generate P/N identification data [*, neighborhood, greeting, 1] indicating that the emotion of everyone becomes positive when greeting in the neighborhood, and perform data organization.

Note that P/N identification data based on general knowledge or P/N identification data based on a questionnaire result regarding preference such as likes and dislikes of the user may be stored in the identification DB 141 in an initial stage for a case where the learning is not performed.

Next, a specific example of the P/N feedback data learning in the feedback DB 142 will be described using the table illustrated in FIG. 8. For example, when an AND condition in each of a situation and an action is removed from data in which a certain user U1 has the same emotion like L2 (ID) [U1, commuting & meeting dog, walking, 1] and L4 (ID) [U1, neighborhood & meeting dog, stopping, 1] illustrated in FIG. 8, it may be appreciated that the emotion becomes positive in a situation of meeting a dog (additional learning of P/N identification data [U1, meeting dog, *, 1] may be performed in the identification DB 141). In this case, since “dog” can be used as feedback (positive reinforcer) for a positive action of the user U1, for example, feedback data [U1, *, *, 1, dog] as indicated in R2 (ID) in FIG. 4 can be generated.

Further, in a case where feedback data [user ID: U2, situation: *, action: *, P/N: 1, FB content: dog] indicating that “dog” can also be used as positive feedback for another user, for example, the user U2 already exists when such P/N feedback data of the user U1 is added, the learning unit 106 may generalize these two feedback data, generate positive reinforcer feedback data [user ID: *, situation: *, action: *, P/N: 1, FB content: dog] indicating that “dog” can be used as positive feedback for everyone, and perform data organization.

Note that feedback data based on general knowledge or feedback data based on a questionnaire result regarding preference such as likes and dislikes of the user may be stored in the feedback DB 142 in an initial stage for a case where the learning is not performed.

The learning in the database has been described above as the second exemplary embodiment. Note that the identification unit 103 may perform the P/N identification based on the emotion recognition result obtained by the emotion recognition unit 105.

### 2-3. Third Exemplary Embodiment (Brain Stimulation)

Feedback for promoting or suppressing an action according to the present embodiment is not limited to a positive or negative reinforcer based on behavior analysis. For example, it is also possible to promote or suppress the action by directly stimulating the brain, like transcranial direct current stimulation (tDCS). With the tDCS, it is possible to promote or suppress perception or action by applying a weak direct current to the head. Specifically, it is known that anodal stimulation promotes a motor function such as jump, and cathodal stimulation suppresses perception such as an itch.

Therefore, in the present exemplary embodiment, it is possible to provide feedback for promoting or suppressing an action of the user by applying anodal stimulation or cathodal stimulation to the brain of the user.

### 2-3-1. Configuration Example

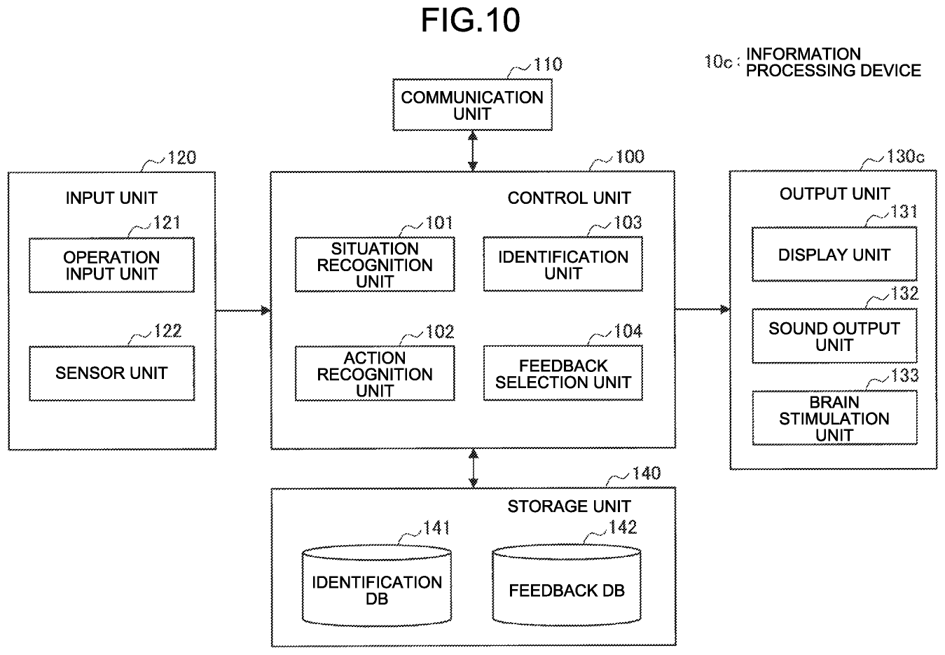

FIG. 10 is a block diagram illustrating an example of a configuration of an information processing device 10c according to the present exemplary embodiment. As illustrated in FIG. 10, the information processing device 10c includes a brain stimulation unit 133 as an output unit 130c.

The brain stimulation unit 133 can provide feedback for promoting an action of the user by applying anodal stimulation to the brain of the user, and provide feedback for suppressing an action of the user by applying cathodal stimulation to the brain of the user. The brain stimulation unit 133 is implemented by, for example, an electrode. The brain stimulation unit 133 may be provided on, for example, a surface of a headband (a band that surrounds an entire circumference of the head, or a band that passes through the temporal region and/or the parietal region) put on the head of the user, the surface coming into contact with a portion of the head between the ears. A plurality of brain stimulation units 133 (electrodes) are arranged so as to come into contact with the sensorimotor areas on both sides of the head of the user when the headband is put on, for example. Further, the information processing device 10c may be implemented by an HMD including such a headband. Note that the shape of the headband or the HMD is not particularly limited.

Other configurations are similar to those of the first exemplary embodiment. Further, the second exemplary embodiment (database learning function) may be applied to the present exemplary embodiment. Further, the identification unit 103 may perform P/N identification based on the emotion recognition result obtained by the emotion recognition unit 105 described in the second exemplary embodiment.

### 2-3-2. Operation Processing

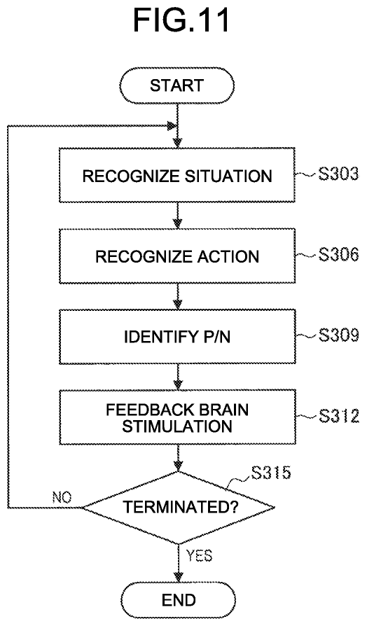

FIG. 11 is a diagram for describing an example of a flow of brain stimulation processing according to the present exemplary embodiment. As illustrated in FIG. 11, first, the information processing device 10c performs situation recognition (Step S303) and action recognition (Step S306). The situation recognition and the action recognition are not limited to the order of the flow illustrated in FIG. 11, and may be performed in the reverse order or in parallel. Further, the situation recognition and the action recognition can be performed continuously/periodically.

Next, the information processing device 10c refers to the identification DB 141 to perform P/N identification based on a situation recognition result and an action recognition result (Step S309).

Next, the information processing device 10c provides brain stimulation feedback according to a P/N identification result (Step S312). Specifically, in the information processing device 10c, the brain stimulation unit 133 provides brain anodal stimulation feedback to promote the action in a case where it is identified that the emotion is positive, and the brain stimulation unit 133 provides brain cathodal stimulation feedback to suppress the action in a case where it is identified that the emotion is negative. For example, the information processing device 10c can simultaneously provide the brain anodal stimulation feedback when it is recognized that a certain user pleasurably greets a person in the neighborhood (recognition of a situation, an action, and a positive emotion) to further promote the greeting action. Further, in the information processing device 10c, the brain stimulation unit 133 can apply the cathodal stimulation when, for example, a certain user sees a cat that is run over by a car at an intersection and does not move any more, and thus becomes sad and has a negative emotion, to suppress the negative emotion. The action recognition unit 102 can determine a gaze or fixation point of the user, and a gaze target by using an outward-facing camera or gaze sensor (such as an infrared sensor) provided in the HMD, and recognize that the user is seeing (perceiving) an animal that is run over by a car and thus does not move.

The above-described processing in Steps S303 to S312 are repeated until the system is terminated (for example, an explicit termination instruction from the user) (Step S315).

### 2-4. Fourth Exemplary Embodiment (Ethical and Legal Considerations)

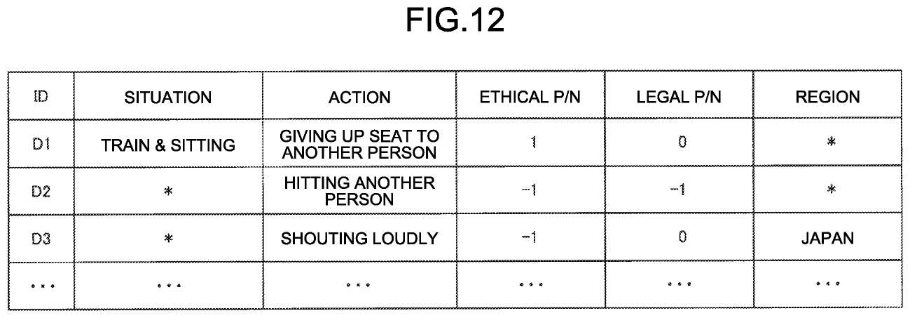

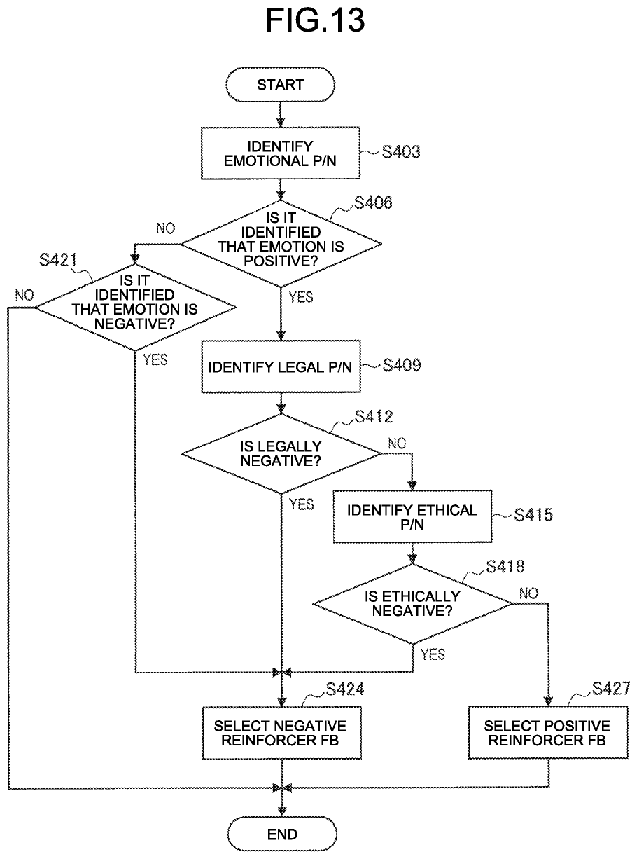

Next, a fourth exemplary embodiment according to the present embodiment will be described with reference to FIGS. 12 and 13. In each embodiment described above, it has been described that feedback for promoting an action by which the emotion of the user becomes positive is provided. However, the action may be reinforced even in a case where the action is ethically or legally unfavorable. In this case, not only the user eventually suffers a disadvantage, but also people around the user will be inconvenienced.

Therefore, in the present exemplary embodiment, feedback for promoting an action is provided in a case where no concern is identified after checking whether or not the action is ethically or legally negative, in addition to performing identification of a positive/negative emotion.

### 2-4-1. Configuration Example

A configuration of an information processing device 10d according to the present exemplary embodiment may be any one of those of the information processing devices 10a, 10b, and 10c according to the first to third exemplary embodiments described above. That is, the present exemplary embodiment can be combined with any one of the first exemplary embodiment, the second exemplary embodiment, or the third exemplary embodiment. Further, the identification unit 103 may perform P/N identification based on the emotion recognition result obtained by the emotion recognition unit 105 described in the second exemplary embodiment.

In the information processing device 10d according to the present exemplary embodiment, the identification unit 103 performs emotional P/N identification, and also performs ethical P/N identification and legal P/N identification. Identification data for the ethical P/N identification and the legal P/N identification are stored in the identification DB 141, for example.

Here, FIG. 12 illustrates an example of a data structure of a database for the ethical P/N identification and the legal P/N identification according to the present exemplary embodiment. As ethical P/N identification data, an action that is generally and socially considered as ethically positive (favorable) or an action that is generally and socially considered as ethically negative (unfavorable) is registered in advance. In addition, as legal P/N identification data, a legally positive (legal) action or a legally negative (illegal) action is registered in advance. Further, since criteria for the ethical P/N identification or legal P/N identification may differ depending on a country or region, a data item may include “region” as illustrated in FIG. 12. Accordingly, in a case where a location of the user can be specified by using a GPS or the like, for example, a country or region where the user is currently located can be known, and thus P/N identification based on the ethics or law of the country or region can be performed.

### 2-4-2. Operation Processing

FIG. 13 is a flowchart illustrating an example of a flow of feedback processing considering the ethical P/N identification and legal P/N identification according to the present exemplary embodiment.

As illustrated in FIG. 13, first, the identification unit 103 of the information processing device 10d refers to the display unit 131 (for example, the table illustrated in FIG. 3) to perform emotional P/N identification based on a situation recognition result and an action recognition result (Step S403).

Next, in a case where it is identified that the emotion is positive (Step S406/Yes), the identification unit 103 refers to the display unit 131 (for example, the table illustrated in FIG. 12) to perform legal P/N identification based on the same situation recognition result and the same action recognition result (Step S409). For example, an action of hitting a person is identified as a legally negative action (problematic action) in any situation.

Next, in a case where it is identified that the action is legally negative (Step S412/No), the identification unit 103 refers to the display unit 131 (for example, the table illustrated in FIG. 12) to perform ethical P/N identification based on the same situation recognition result and the same action recognition result (Step S415). For example, an action of shouting loudly is identified as an ethically negative action (problematic action), even in a case where the action is not legally negative.

The ethical P/N identification (Step S409) and the legal P/N identification (Step S415) described above can be performed at least when it is identified that the emotion is positive in the emotional P/N identification (Step S403). For example, even in a case where the action of shouting and the action of hitting make the user have a positive emotion, these actions are ethically or legally negative actions and thus should not be promoted. On the other hand, in a case where the action of shouting or hitting makes the user have a negative emotion, FB for suppressing the action is provided without performing the ethical P/N identification or legal P/N identification. Therefore, in the flowchart illustrated in FIG. 13, the ethical P/N identification or legal P/N identification is performed in a case where it is identified that the emotion is positive.

Then, in a case where it is identified that the emotion is negative (Step S421/Yes), in a case where it is identified that the emotion is positive (Step S406/Yes), but it is identified that the action is legally negative (Step S412/Yes), or in a case where it is identified that the action is ethically negative (Step S418/Yes), the feedback selection unit 104 selects negative reinforcer FB in order to suppress the action (Step S424).

On the other hand, in a case where it is identified that the emotion is positive (Step S421/Yes), and it is identified that the action is not legally or ethically negative (is positive) (Step S412/No and Step S418/No), the feedback selection unit 104 selects positive reinforcer FB as FB for the action (Step S427).

As a result, in the information processing system according to the present exemplary embodiment, for example, even in a case where the user U2 is a person who feels pleasure in violence such as hitting a person, and the user U2 involuntarily hits a person who has bumped into the user U2 on the train and feels pleasure, since such an action is not an ethically or legally favorable action, the action is not promoted, and it is possible to suppress the action with the negative reinforcer FB. Note that recognition of an action such as hitting a person is performed by, for example, analyzing sensing data of an acceleration sensor (an example of the sensor unit 122) provided in an HMD (information processing device 10d) worn by the user U2 and a video of a camera (an example of the sensor unit 122) provided in the HMD. Specifically, for example, the action recognition unit 102 can recognize the action in which the user hits the other person in a case where the action recognition unit 102 analyzes that a change in acceleration peculiar to the action of hitting a person is shown in the sensing data of the acceleration sensor, and analyzes from the camera video that the arm extending from the front side (user side) comes into contact with the other person.

### 2-5. Fifth Exemplary Embodiment (Reframing)

Next, a fifth exemplary embodiment according to the present embodiment will be described. In the present exemplary embodiment, reframing that presents a positive interpretation with respect to an action that makes the user have a negative emotion is performed, such that it is possible to reduce the negative emotion of the user, regard the action as an action in a positive situation, and promote the action.

A specific example of a reframing effect will be described below. For example, a certain user luckily sat in a seat on a train on the way home after a long walk, and when the user stood up, thinking that he/she had arrived at the nearest station, the user realized that there is still one more stop. When the user looked back at his/her seat to sit down again, the other person was already sitting on the seat, and thus the user suddenly felt tired and felt a little depressed. The user had no choice but to stand as he/she is, and at this time, the user heard a voice of the agent saying that “you stood up by mistake, but you did a good thing because you gave up your seat to another person” from an earphone speaker of an HMD or the like. From what the agent said, the user can think of things in a positive way, for example, “maybe I did a good thing”, and an effect that the feeling of depression disappears and the feeling gets lighter can be expected.

Here, the system recognizes a situation where the user is on a train based on, for example, location information of a GPS of the HMD (information processing device 10e) worn by the user and acceleration sensor information. Further, the information processing device 10e analyzes the acceleration sensor information and recognizes (performs action recognition) that the user transitions from a sitting state to a standing state. Next, the information processing device 10e recognizes that the user does not get off the train, but remains standing. Further, the information processing device 10e recognizes that an emotion of the user is in a negative state based on a facial expression of the user acquired by a camera, pulse data, and the like. Then, the information processing device 10e refers to the feedback DB 142 based on the situation where the user was sitting in the seat on the train, the action of standing up from the seat but not getting off at the station, and the identification result indicating that the emotion is negative, and provides, in a case where there is corresponding reframing FB (for example, the phrase as described above), the reframing FB (for example, outputting a voice of the agent from the sound output unit 132).

### 2-5-1. Configuration Example

A configuration of the information processing device 10e according to the present exemplary embodiment may be any one of those of the information processing devices 10a, 10b, 10c, and 10d according to the first to fourth exemplary embodiments described above. That is, the present exemplary embodiment can be combined with any one of the first exemplary embodiment, the second exemplary embodiment, the third exemplary embodiment, or the fourth exemplary embodiment. Further, the identification unit 103 may perform P/N identification based on the emotion recognition result obtained by the emotion recognition unit 105 described in the second exemplary embodiment.

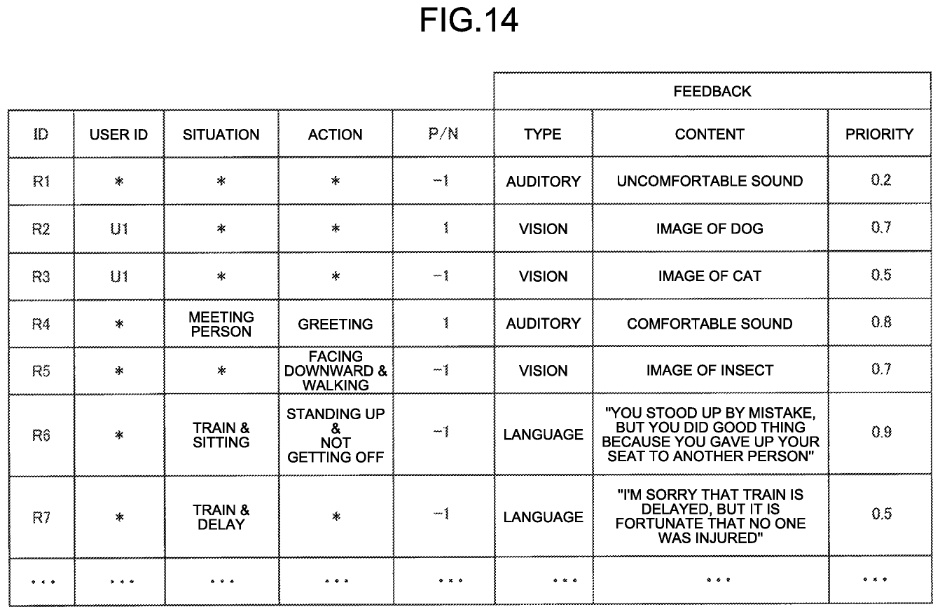

In the information processing device 10e according to the present exemplary embodiment, the feedback selection unit 104 refers to the feedback DB 142 to select feedback (action promoting FB, action suppressing FB, or reframing FB) to the user based on an emotional P/N identification result. Here, FIG. 14 illustrates an example of a data structure of the feedback DB 142 for selection of feedback including the reframing FB according to the present exemplary embodiment.

Among feedback data illustrated in FIG. 14, examples of reframing FB corresponding to R6 (ID) and R7 (ID) are described. Both FB types are a language, and basically, it is assumed that a positive interpretation in which an evaluation standard is changed is presented to an interpretation of the user who has a negative emotion. Such a change in evaluation standard can include a change from an egoistic evaluation to an altruistic evaluation, a change from a subjective evaluation to an objective evaluation, a shift of a relative evaluation standard, or the like.

For example, when the train is delayed and the user may be late for work, and thus the emotion of the user becomes negative (frustrated, worried, or the like), the information processing device 10e may use the feedback DB 142 to present an altruistic positive interpretation like “I'm sorry that the train is delayed, but it is fortunate that no one was injured” in a case where information indicating that the delay is not caused by an injury accident can be acquired from train delay information. Alternatively, when someone bumps into the user or is stepped on the user on the train and thus the user has a negative emotion, the information processing device 10e uses the feedback DB 142 to present an altruistic positive interpretation like “it is fortunate that he/she did not fall”.

In order to make the altruistic positive interpretation more acceptable to the user, the information processing device 10e may present the altruistic positive interpretation only when the other person is a weak person such as an old person, a child, an injured person, or a pregnant woman.

In addition, when the user has a negative emotion “it is still dirty” after looking at a place cleaned by another person, the information processing device 10e may perform the reframing in which a change from a subjective evaluation to an objective evaluation is performed, for example, presenting an objective evaluation such as a cleanliness level, or promoting comparison before and after the cleaning, rather than comparison with a result of cleaning performed by the user.

The information processing device 10e according to the present embodiment may extract, based on the feedback data registered in advance in the feedback DB 142 as described above, reframing FB of which conditions of a situation, an action, and an emotion match, and present the reframing FB to the user. Alternatively, the information processing device 10e can also automatically generate reframing depending on the situation, or automatically add (learn) the data in the feedback DB 142. Such automatic generation of the reframing will be described later. Note that the information processing device 10e may have at least the reframing FB function among the action promoting FB function, the action suppressing FB function, and the reframing FB function.

### 2-5-2. Operation Processing

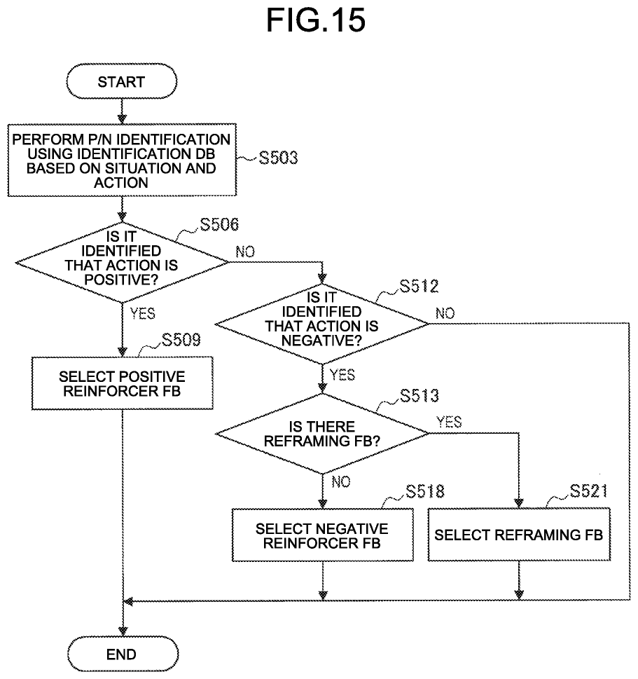

FIG. 15 is a flowchart illustrating an example of a flow of feedback processing including the reframing according to the present exemplary embodiment.

As illustrated in FIG. 15, the identification unit 103 of the information processing device 10e refers to the display unit 131 (for example, the table illustrated in FIG. 3) to perform emotional P/N identification based on a situation recognition result and an action recognition result (Step S503). Alternatively, the identification unit 103 may perform the emotional P/N identification based on a recognition result obtained by the emotion recognition unit 105.

Next, in a case where it is identified that the emotion is positive (Step S506/Yes), the feedback selection unit 104 refers to the feedback DB 142 and selects corresponding positive reinforcer FB (Step S509). For example, R2 (ID), R4 (ID), or the like in the table illustrated in FIG. 14 corresponds to the case.

On the other hand, in a case where it is identified that the emotion is negative (Step S512/Yes), the feedback selection unit 104 refers to the feedback DB 142 and determines whether or not there is reframing FB corresponding to the recognized situation, action, and emotion (Step S515).

Next, in a case where there is reframing FB (Step S515/Yes), the feedback selection unit 104 selects the corresponding reframing FB (Step S521). For example, R6 (ID), R7 (ID), or the like in the table illustrated in FIG. 14 corresponds to the case.

On the other hand, in a case where there is no reframing FB (Step S515/No), the feedback selection unit 104 selects a corresponding negative reinforcer FB (Step S518). For example, R1 (ID), R3 (ID), R5 (ID), or the like in the table illustrated in FIG. 14 corresponds to the case.

### 2-5-3. Adding Response Showing Empathy

In the example described above, the system performs the reframing without mentioning the negative emotion of the user. However, a highly sympathetic response to the negative emotion may be added to more effectively suppress the negative emotion and achieve a change to a positive interpretation. For example, a condition of the reframing FB is further limited, and a response showing empathy to the negative emotion is added to a content of the corresponding reframing FB.

More specifically, for example, in the table illustrated in FIG. 14, a condition of “situation” of R6 (ID) is set to “tired & train & sitting” and a content of the reframing FB is set to “You must be tired from all the hard work today. But you did a good thing to give up the seat to another person”. By doing so, it is possible to return a highly sympathetic response to the negative emotion of the user. As for the situation “tired”, for example, the situation recognition unit 101 may calculate calorie consumption of the day from the acceleration sensor data, compare the calorie consumption with an average (for example, average calorie consumption of the user for one day), and determine that the user is tired in a case where the calorie consumption exceeds a predetermined threshold value (for example, 120%).

### 2-5-4. Automatic Generation of Reframing

The information processing device 10e according to the present embodiment can apply the configuration of the second exemplary embodiment described above, and perform learning in the feedback DB 142 including the reframing FB by using, for example, the learning DB 143.

Change to Altruistic Interpretation

For example, the information processing device 10e can automatically generate the reframing FB in a case where corresponding actions such as “dropping/leaving-picking up/using”, “standing-sitting”, and “using-not using” are specified in advance as a rule, and a situation where the corresponding actions are taken and emotions are opposite in the same situation (train, conference room, home, company, or the like) is stored in the learning DB 143.

For example, among the data accumulated in the learning DB 143 illustrated in FIG. 8, L6 (ID) [U1, home & chocolate, eating, 1] and L7 (ID) [U2, home & cake, leaving, −1] correspond to corresponding actions “leaving” and “using (eating)”, and the emotions are opposite. Such data suggests that although someone had left his/her food and thus his/her emotion becomes negative, another person (such as a family member) may eat the left food and thus his/her emotion may become positive. The learning unit 106 can generate, based on these data, a content (text) of the reframing FB like [user ID: *, situation: [food], action: leaving, P/N: −1, FB type: language, FB content: “although it is sad that you has left the [food], a person who finds it may be happy to eat the [food]”.

Further, among the data accumulated in the learning DB 143 illustrated in FIG. 8, L8 (ID) [U3, company & umbrella, leaving, −1] and L9 (ID) [U4, company & umbrella, using, 1] correspond to corresponding actions “leaving” and “using”, and the emotions are opposite. Such data suggests that although someone had left his/her umbrella and thus his/her emotion becomes negative, another person (such as a co-worker or family member, assuming an environment in which sharing can be performed to some extent) may use the left umbrella and thus his/her emotion may become positive. The learning unit 106 can generate, based on these data, a content (text) of the reframing FB like [user ID: *, situation: [object], action: leaving, P/N: −1, FB type: language, FB content: “although it is sad that you has left the [object], a person who finds it may be happy to [be able to use] the [object]”.

Alternatively, the information processing device 10e can generate a content (text) of the reframing FB by extracting, from a social media on which texts or voices are posted by multiple users, a post including an interpretation on a situation where emotions are opposite in the same situation, and entering the post in a database. For example, in a case where a post including “I couldn't sit on a train (situation)-I wanted to sit down because I've had a hard day (interpretation)-it's annoying (emotion)” and a post including “I couldn't sit on a train (situation)-but some people seemed to sit down (interpretation)-I'happy for them (emotion)” are extracted from a social media, the information processing device 10e presents, to a user who has a negative emotion, an interpretation of another user who has a positive emotion in the similar situation. Specifically, for example, the information processing device 10e can present, based on the posts collected from the social media, a positive interpretation like “but some people seemed to sit down” when the user has a negative emotion because he/she cannot sit on the train.

In addition, the information processing device 10e may acquire a user evaluation (explicit evaluation, emotion recognition result (whether or not the emotion is actually turned into positive), or the like) with respect to the reframing FB to learn effective reframing.

Presentation of Relative Evaluation

Further, in a case where keywords related to “comparison” such as “strong, high, large, and clean” are stored as a knowledge database in advance, the information processing device 10e can perform the reframing by presenting a relative evaluation when the user utters these keywords and has a negative emotion. A reframing content can be generated by extracting, for example, from a social media, posts that include opposite emotions for similar situations including a keyword related to “comparison” and evaluations (interpretations) thereof. For example, in a case where a post including “the child's clothing is dirty (situation [evaluation target])-washing becomes difficult (evaluation)-angry (emotion)” and a post including “the child's clothing is dirty (situation [evaluation target])-the child is having fun (evaluation)-happy (emotion)” are extracted, it is possible to present a positive interpretation in which an evaluation standard is changed to a user who has a negative emotion for a similar relative evaluation. Specifically, for example, the information processing device 10e can present, based on the posts collected from the social media, a positive interpretation like “but the child is having fun!” when the user has a negative emotion because the child's clothing is dirty.

### 2-6. Sixth Exemplary Embodiment (Example of Application to Small Communities)

The information processing system according to the present embodiment can also provide a positive action promoting FB, a negative action suppressing FB, and a reframing FB to a small community such as a family, a company, a department, a school class, or a neighborhood association. In a case of the small community, members or places are limited, which enables more accurate situational recognition and action recognition.

As an example, assuming a situation where a child is cleaning a room, a cleaning action is promoted and an action such as playing during the cleaning is suppressed. In addition, when the child did not properly clean the room, the parent often scolds the child, which rather suppresses the cleaning action of the child. Therefore, the reframing is performed to turn such a negative emotion of the parent.

### 2-6-1. Configuration Example

A configuration of an information processing device 10f according to the present exemplary embodiment may be any one of those of the information processing devices 10a to 10e according to the first to fifth exemplary embodiments described above. That is, the present exemplary embodiment can be combined with any one of the first exemplary embodiment, the second exemplary embodiment, the third exemplary embodiment, the fourth exemplary embodiment, or the fifth exemplary embodiment. Further, the identification unit 103 may perform P/N identification based on the emotion recognition result obtained by the emotion recognition unit 105 described in the second exemplary embodiment.

In the information processing device 10f according to the present exemplary embodiment, the feedback selection unit 104 refers to the feedback DB 142 to select feedback (action promoting FB, action suppressing FB, or reframing FB) to the user (child or parent) based on an emotional P/N identification result. Further, the feedback selection unit 104 may select feedback to the user (child or parent) in consideration of the ethical P/N identification or legal P/N identification described in the fourth exemplary embodiment.

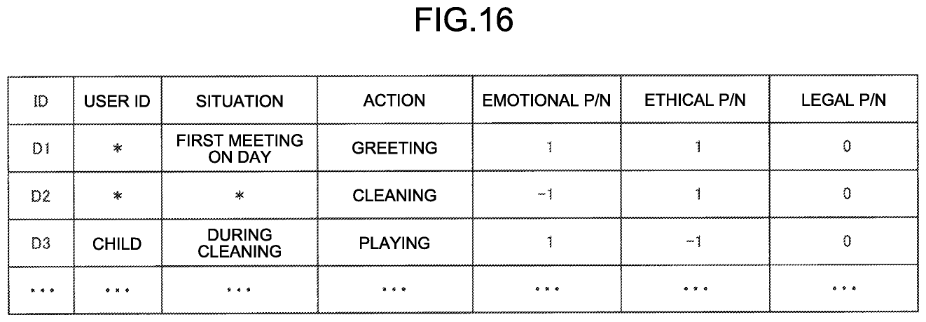

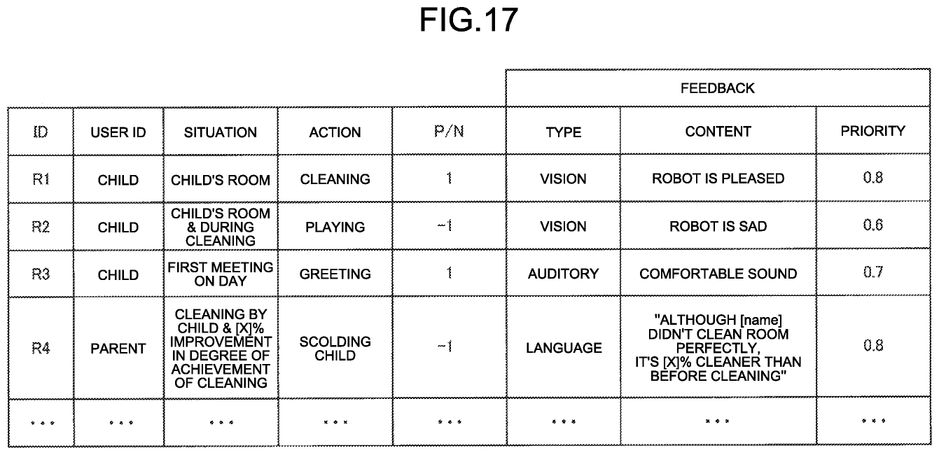

Here, FIG. 16 illustrates an example of a data structure of a database for P/N identification in consideration of ethical P/N identification and legal P/N identification according to the present exemplary embodiment. For example, as indicated in D3 (ID) in FIG. 16, for example, an emotional P/N is positive because playing while cleaning is enjoyable for the child, but such an action is ethically negative (unfavorable). Further, FIG. 17 illustrates an example of a data structure of the feedback DB 142 for selection of feedback including the reframing FB according to the present exemplary embodiment. For example, as indicated in R1 (ID) in FIG. 17, since cleaning of a child's room by the child corresponds to a positive state, FB for promoting the cleaning action of the child, such as a robot being happy, is registered.

### 2-6-2. Operation Processing

Next, specific operation processing according to the present exemplary embodiment will be described. In the present exemplary embodiment, for example, the operation processing illustrated in FIG. 15 can be applied to perform action promotion/suppression processing based on P/N identification and reframing processing.

For example, in the information processing device 10e, the situation recognition unit 101 recognizes that a parent and a child are in a child's room from a video of a camera installed in each room of a house. Furthermore, in the information processing device 10e, the action recognition unit 102 recognizes that the child is cleaning from the video of the camera.

Next, the identification unit 103 of the information processing device 10e refers to the identification DB 141 to perform P/N identification with respect to the cleaning of the child's room by the child (Step S503 illustrated in FIG. 15). Specifically, for example, as indicated in D2 (ID) in FIG. 16, it is determined that the cleaning action is emotionally negative (emotional P/N identification: −1), but is legally unproblematic (legal P/N identification: 0), and ethically positive (1; ethical P/N identification; 1). In this case, the identification unit 103 gives priority to the ethical identification and identifies that the cleaning action is positive.

Next, since it is identified that P/N of the action in which the child cleans the child's room is (ethically) positive (S506/Yes illustrated in FIG. 15), the feedback selection unit 104 of the information processing device 10e transmits FB for promoting the action, for example, a control signal to cause, for example, a robot placed in the child's room to show a joyful gesture, according to R1 (ID) illustrated in FIG. 17 (Step S509 illustrated in FIG. 15). It can be expected that the child spontaneously and often performs the cleaning action because the robot will be pleased when cleaning.

On the other hand, when it is recognized that the child stopped cleaning and started playing with a toy, the identification unit 103 of the information processing device 10e determines that playing during cleaning is ethically negative according to D3 (ID) in FIG. 16 (Step S503 illustrated in FIG. 15).

In this case, since it is identified that P/N of the action in which the child plays during cleaning is (ethically) negative (S512/Yes illustrated in FIG. 15), the feedback selection unit 104 of the information processing device 10e transmits FB for suppressing the action, for example, a control signal to cause, for example, a robot (for example, a dog robot that the child likes) placed in the child's room to show a sad gesture, according to R2 (ID) illustrated in FIG. 17 (Step S518 illustrated in FIG. 15). It can be expected that the child tries not to play during cleaning, because the robot will be sad if playing during cleaning.

Next, presentation of reframing in a case where the parent feels that the room is still messed up after the child finishes cleaning and scolds the child by saying that “it's not clean at all!” will be described. The situation recognition unit 101 of the information processing device 10e can analyze a video of a camera provided in the child's room to recognize a degree of messiness of the child's room before cleaning (such as a state where things are scattered) and a state of the room after cleaning. The situation recognition unit 101 can compare videos before and after cleaning by the child and calculate a degree of achievement of cleaning (for example, a decrease in the number of scattered things, an increase rate of a floor area in which nothing is placed, or the like).

Further, the action recognition unit 102 of the information processing device 10e can analyze an uttered voice of the parent collected from a microphone and recognize that the parent is scolding the child. Further, the emotion recognition unit 105 of the information processing device 10e can identify that the emotion of the parent is negative based on a facial expression of the parent analyzed from a video of a camera installed in the room, a pulse rate acquired from a smart band worn by the mother, or a voice recognition result of uttered voice data acquired from a microphone installed in the room or a microphone of an HMD worn by the parent.

As such, in a case where the parent takes an action of scolding the child and the emotion of the parent becomes negative in a situation where the child has cleaned, the feedback selection unit 104 of the information processing device 10e refers to the feedback DB 142 and provides, in a case where there is reframing FB, the reframing FB (Step S515/Yes and S521 illustrated in FIG. 15). For example, according to R4 (ID) illustrated in FIG. 17, in a case where, for example, a degree of achievement of cleaning is equal to or more than a predetermined value, the feedback selection unit 104 of the information processing device 10e presents, as reframing, an objective evaluation like “although XX didn't clean the room perfectly, it's XX % cleaner than before cleaning”. By doing so, a parent's anger subsides, and a result of cleaning can be objectively grasped, such that it is possible to praise the child and teach areas to be improved. Further, the child does not remain scolded for cleaning, and thus it is possible to prevent the cleaning action from being suppressed.

### 3. Conclusion

As described above, in the information processing system according to the embodiment of the present disclosure, it is possible to make a state of a user better according to an emotion of the user and improve quality of life. The information processing system according to the present embodiment can perform at least one of the action promoting FB function, the action suppressing FB function, or the reframing FB function described above.

Although the preferred embodiment of the present disclosure has been described above in detail with reference to the appended drawings, the present technology is not limited to such an example. It is obvious that a person with an ordinary skill in a technological field of the present disclosure could conceive of various modifications or corrections within the scope of the technical ideas described in the appended claims, and it should be understood that such modifications or corrections also fall within the technical scope of the present disclosure.

For example, a computer program can also be created for causing hardware such as a CPU, a ROM, and a RAM built in the information processing device 10 to function as the information processing device 10. Further, a computer-readable storage medium storing the computer program is also provided.

Furthermore, the effects described in the present specification are merely illustrative or exemplary and are not restrictive. That is, the technology according to the present disclosure can exhibit other effects obvious to those skilled in the art from the description of the present specification in addition to or in place of the above-described effects.

Note that the present technology can also have the following configurations.

(1)

An information processing system comprising:

a control unit that

estimates whether a user is positive or negative, and

has any one of a function of promoting an action of the user when it is estimated that the user is positive,

a function of suppressing an action of the user when it is estimated that the user is negative, or

a function of presenting a positive interpretation on a situation or action of the user when it is estimated that the user is negative.

(2)

The information processing system according to (1), wherein

the control unit

estimates whether the user is positive or negative due to any one of a situation or an action of the user based on data indicating a relationship between any one of a situation or an action and a positive or negative emotional state for the situation or action, the data being learned in advance.

(3)

The information processing system according to (1), wherein

the control unit

estimates an emotion of the user to estimate whether the user is positive or negative.

(4)

The information processing system according to (3), wherein the control unit estimates whether the emotion of the user is positive or negative based on sensing data of the user.

(5)

The information processing system according to any one of (1) to 4, wherein

the control unit

controls an agent that interacts with the user, and

the agent provides positive feedback to the user, as the function of promoting an action of the user when it is estimated that the user is positive.

(6)

The information processing system according to (5), wherein

the control unit

presents, to the user, at least one of a predetermined comfortable sound, image, or vibration as the positive feedback.

(7)

The information processing system according to any one of (1) to (6), wherein

the control unit

applies anodal stimulation to a brain of the user, as the function of promoting an action of the user when it is estimated that the user is positive.

(8)

The information processing system according to any one of (1) to (7), wherein

the control unit

controls an agent that interacts with the user, and

the agent provides negative feedback to the user, as the function of suppressing an action of the user when it is estimated that the user is negative.

(9)

The information processing system according to (8), wherein

the control unit

presents, to the user, at least one of a predetermined uncomfortable sound, image, or vibration as the negative feedback.

(10)

The information processing system according to any one of (1) to (9), wherein

the control unit

applies cathodal stimulation to a brain of the user, as the function of suppressing an action of the user when it is estimated that the user is negative.

(11)

The information processing system according to any one of (1) to (10), wherein

the control unit

provides, in a case where an action of the user is legally and ethically unproblematic when it is estimated that the user is positive, positive feedback for promoting the action.

(12)

The information processing system according to any one of (1) to (11), wherein

the control unit

controls an agent that interacts with the user, and

performs, when it is estimated that the user is negative, a control to cause the agent to present, in a case where a situation or action of the user and a text representing a positive interpretation on the situation or action are stored, the text representing the positive interpretation.

(13)

The information processing system according to (12), wherein

the control unit

performs a control to generate the text representing the positive interpretation based on information indicating opposite emotions for similar or corresponding actions in similar situations and present the generated text to the user.

(14)

An information processing method, by a processor, comprising:

estimating whether a user is positive or negative; and

performing any one of a function of promoting an action of the user when it is estimated that the user is positive,

a function of suppressing an action of the user when it is estimated that the user is negative, or

a function of presenting a positive interpretation on a situation or action of the user when it is estimated that the user is negative.

(15)

A recording medium recording a program for causing a computer to function as

a control unit that

estimates whether a user is positive or negative, and

has any one of a function of promoting an action of the user when it is estimated that the user is positive,

a function of suppressing an action of the user when it is estimated that the user is negative, or

a function of presenting a positive interpretation on a situation or action of the user when it is estimated that the user is negative.

### REFERENCE SIGNS LIST

10, 10a to 10f INFORMATION PROCESSING DEVICE
100 CONTROL UNIT
101 SITUATION RECOGNITION UNIT
102 ACTION RECOGNITION UNIT
103 IDENTIFICATION UNIT
104 FEEDBACK SELECTION UNIT
105 EMOTION RECOGNITION UNIT
106 LEARNING UNIT
110 COMMUNICATION UNIT
120 INPUT UNIT
121 OPERATION INPUT UNIT
122 SENSOR UNIT
130 OUTPUT UNIT
131 DISPLAY UNIT
132 SOUND OUTPUT UNIT
133 BRAIN STIMULATION UNIT
140 STORAGE UNIT
141 IDENTIFICATION DB
142 FEEDBACK DB
143 LEARNING DB

1. An information processing system comprising:
a control unit that
estimates whether a user is positive or negative, and
has any one of a function of promoting an action of the user when it is estimated that the user is positive,
a function of suppressing an action of the user when it is estimated that the user is negative, or
a function of presenting a positive interpretation on a situation or action of the user when it is estimated that the user is negative.

2. The information processing system according to claim 1, wherein
the control unit
estimates whether the user is positive or negative due to any one of a situation or an action of the user based on data indicating a relationship between any one of a situation or an action and a positive or negative emotional state for the situation or action, the data being learned in advance.

3. The information processing system according to claim 1, wherein
the control unit
estimates an emotion of the user to estimate whether the user is positive or negative.

4. The information processing system according to claim 3, wherein the control unit estimates whether the emotion of the user is positive or negative based on sensing data of the user.

5. The information processing system according to claim 1, wherein
the control unit
controls an agent that interacts with the user, and
the agent provides positive feedback to the user, as the function of promoting an action of the user when it is estimated that the user is positive.

6. The information processing system according to claim 5, wherein
the control unit
presents, to the user, at least one of a predetermined comfortable sound, image, or vibration as the positive feedback.

7. The information processing system according to claim 1, wherein
the control unit
applies anodal stimulation to a brain of the user, as the function of promoting an action of the user when it is estimated that the user is positive.

8. The information processing system according to claim 1, wherein
the control unit
controls an agent that interacts with the user, and
the agent provides negative feedback to the user, as the function of suppressing an action of the user when it is estimated that the user is negative.

9. The information processing system according to claim 8, wherein
the control unit
presents, to the user, at least one of a predetermined uncomfortable sound, image, or vibration as the negative feedback.

10. The information processing system according to claim 1, wherein
the control unit
applies cathodal stimulation to a brain of the user, as the function of suppressing an action of the user when it is estimated that the user is negative.

11. The information processing system according to claim 1, wherein
the control unit
provides, in a case where an action of the user is legally and ethically unproblematic when it is estimated that the user is positive, positive feedback for promoting the action.

12. The information processing system according to claim 1, wherein
the control unit
controls an agent that interacts with the user, and
performs, when it is estimated that the user is negative, a control to cause the agent to present, in a case where a situation or action of the user and a text representing a positive interpretation on the situation or action are stored, the text representing the positive interpretation.

13. The information processing system according to claim 12, wherein
the control unit
performs a control to generate the text representing the positive interpretation based on information indicating opposite emotions for similar or corresponding actions in similar situations and present the generated text to the user.

14. An information processing method, by a processor, comprising:
estimating whether a user is positive or negative; and
performing any one of a function of promoting an action of the user when it is estimated that the user is positive,
a function of suppressing an action of the user when it is estimated that the user is negative, or
a function of presenting a positive interpretation on a situation or action of the user when it is estimated that the user is negative.

15. A recording medium recording a program for causing a computer to function as
a control unit that
estimates whether a user is positive or negative, and
has any one of a function of promoting an action of the user when it is estimated that the user is positive,
a function of suppressing an action of the user when it is estimated that the user is negative, or
a function of presenting a positive interpretation on a situation or action of the user when it is estimated that the user is negative.
